(19) United States
(12) Patent Application Publication
(10) Pub. No.: US 2023/0122799 A1
(43) Pub. Date: Apr. 20, 2023

(54) NANO-CERAMIC COATING MAGNIFIED DENTAL MIRROR

(71) Applicant: Imam Abdulrahman Bin Faisal University, Dammam (SA)

(72) Inventors: Fatimah Mohammed AL SAFER, Dammam (SA); Ahmed Maniallah AL THOBITY, Dammam (SA)

(73) Assignee: Imam Abdulrahman Bin Faisal University, Dammam (SA)

(21) Appl. No.: 17/501,140

(22) Filed: Oct. 14, 2021

(51) Int. Cl. A61B 1/253

(52) U.S. Cl. CPC A61B 1/253; G02B 27/0006

(57) ABSTRACT

The present disclosure provides a dental mirror having a handle and a mirror head coupled to a first end of the handle. The mirror head includes a double-sided mirror having a nano-ceramic coating that is hydrophobic and transparent. The nano-ceramic coating includes more than 80 wt % of silicon dioxide nanoparticles having an average diameter of less than 100 nm. The dental mirror also includes means for rotating the mirror head and a first button configured to control rotation of the mirror head.

### BACKGROUND

### Technical Field

The present disclosure is directed to dental instruments and, specifically, to a dental mirror.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

During dental treatment procedures, dentists or other dental professionals generally use a dental mirror. Conventionally, dental mirrors include a small circular piece of reflective glass held in a frame and affixed to an elongated handle that is held by the dentist or another dental professional performing the dental treatment procedure. The dental mirror is used in tandem with other dental instruments for viewing of a region of the mouth to be treated. Typically, dental mirrors are subjected to debris generated during the dental treatment procedure, cleaning fluid being supplied into the mouth, and/or the patient's saliva. Additionally, the patient's breath through the mouth may also fog the dental mirror. Accumulation of such debris and fog on the dental mirror compels the dentist to pause the dental treatment procedure in regular intervals to clean the dental mirror, which disrupts the dentist's work, besides being unhygienic and prolonging duration of the dental treatment procedure. Further, withdrawing the dental mirror from the patient's mouth, wiping the dental mirror, and re-inserting the dental mirror back into the patient's mouth (which requires re-orientation by the dentist and re-acquiring “target” in the patient's mouth) may consume only 10 to 15 seconds overall for experienced dentists, but repeating these steps for 50 to 100 times in a day may have significant impact on the stress level of the dentist. For instance, such cumulative unproductive time may add up to about 1500 seconds, or more than 25 minutes in a day for the dentist.

Based on the experience level of the dentist, a lack of skill to locate an appropriate region for treatment may increase the dependency on the dental mirror, and lack of skill to appropriately orient the dental mirror at the identified region may prolong the duration of the dental treatment procedure. For example, if the dental treatment procedure is required to be performed towards the posterior teeth, the dentist may need to rely completely on the dental mirror to view the posterior teeth. Owing to such a tedious and repeated procedure of cleaning the dental mirror, more often than not, the dentist may resist withdrawal of the dental mirror even when the dentist is unable to clearly view the region due to accumulation of the debris and fog. When visibility of the region in the dental mirror is impaired towards the end of a particular step in the dental treatment procedure, the dentist may tend to complete the step with impaired vision instead of prolonging the dental treatment procedure by repeated cleaning of the dental mirror. Such an approach may have adverse dental consequences on the patient.

Several attempts have been made to provide dental mirrors which can be cleaned using different techniques and/or methods. For instance, CN205286301U discloses a double-faced and anti-fog mouth mirror having a flat mirror and a magnifying glass. The mouth mirror includes a ceramic thermostat and a heating element arranged between the flat mirror and the magnifying glass, which prevents fog formation by heating from within.

JP2008113809A discloses a titanium oxide layer used for coating a dental mirror. This titanium oxide layer is hydrophobic due to its photocatalytic activity and may be sterilized by irradiating weak UV light.

GB2550219A discloses a dental device including an elongated handle and a head, with the head and the handle connected at a neck. The head includes two mirror faces, namely a first mirror face and a second mirror face. The neck permits variation in arrangement of the faces with respect to the handle.

JP2005232066A discloses nano-ceramic microparticles having a ceramic microparticle main body which consists of a silicon carbide and/or its unreacted substance. The nano-ceramic microparticles may be used for a mirror as an anti-condensation agent.

U.S. Pat. No. 9,028,958B2 discloses a super hydrophilic nanostructure that may be used as a self-cleaning coating for a dental mirror. The super hydrophilic nanostructure includes aggregate clusters having dimensions of about 150 nanometers to 5 microns, which includes porous clusters having dimensions of about 20 nanometers to 600 nanometers, which further includes nanoparticles having dimensions of about 1 nanometer to 100 nanometers.

In the paper titled “Effect of Nano Titanium Dioxide on Self-Cleaning and Antibacterial Properties of Dental Mirrors”, Sara et al. studied the effect of nano TiO₂ on self-cleaning of dental mirrors and found that nano TiO₂ coated mirrors did not have any superiority over the conventional dental mirrors in terms of self-cleaning properties.

Each of the aforementioned references suffers from one or more drawbacks hindering their adoption. For example, the double-faced and anti-fog mouth mirror of CN205286301U includes a ceramic thermostat and heating element that may need additional supporting structures which add to the weight of the mouth mirror device making it bulky. The use of titanium oxide layer for dental mirrors as per JP2008113809A requires that a hydrophilic film containing silica or alumina to be formed between the titanium oxide layer and a mirror glass to enhance the antifogging property of the titanium oxide layer. GB2550219A fails to disclose any self-cleaning feature for the disclosed dental device; and further does not explicitly describe any mechanism that aid switching between the first mirror face and the second mirror face.

Accordingly, it is one object of the present disclosure to provide a dental mirror which includes self-cleaning characteristics, and which may be easily switched to have either a first side or a second side thereof facing the oral cavity while performing the dental treatment procedure.

### SUMMARY

In an exemplary embodiment, a dental mirror is described. The dental mirror includes a handle and a mirror head coupled to a first end of the handle. The mirror head includes a double-sided mirror having a nano-ceramic coating that is hydrophobic and transparent. The nano-ceramic coating includes more than 80 wt % of silicon dioxide nanoparticles having an average diameter of less than 100 nm. The dental mirror also includes means for rotating the mirror head and a first button configured to control rotation of the mirror head. In some embodiments, the mirror head is removable.

In some embodiments, the nano-ceramic coating is formed by treating the double-sided mirror with KubeBond GlassShield. In some embodiments, the double-sided mirror has a first side providing a normal view and a second side providing a magnified view. In some embodiments, the second side provides four times magnification. In some embodiments, the double-sided mirror has a diameter in a range of about 20 mm to about 24 mm.

In some embodiments, the dental mirror further includes a lighting element for producing light and a second button configured to turn on and off the lighting element. In some embodiments, the lighting element is positioned on the handle and faces the mirror head.

In some embodiments, the dental mirror further includes a base that is wider than the handle and affixed to a second end of the handle. In some embodiments, the base is 1.5 to 2.3 times as wide as the handle. In some embodiments, the base has a diameter of about 9 mm to about 14 mm, and the handle has a diameter of about 5 mm to about 7 mm. In some embodiments, an overall length of the base and the handle is in a range of about 130 mm to about 150 mm. In some embodiments, masses of the mirror head and the base are balanced so that the dental mirror has a center of gravity which is closer to the base than to the mirror head.

In some embodiments, the dental mirror further includes a battery bank space defined in the base.

In some embodiments, the mirror head includes a shaft arranged between the double-sided mirror and the handle. In some embodiments, the shaft is coupled to the first end of the handle such that the shaft is rotatable and removable. Further, the first button is configured to control rotation of the shaft. In some embodiments, the shaft rotates 180 degrees per rotation to switch sides for the double-sided mirror.

In some embodiments, the shaft and the double-sided mirror are placed at an angle such that the shaft and the double-sided mirror are non-coplanar. In some embodiments, the double-sided mirror is manually rotatable on the shaft for adjusting the angle.

In some embodiments, the dental mirror further includes a rubber frame along a circumference of the double-sided mirror.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a perspective view of a dental mirror according to an embodiment of the present disclosure;

FIG. 1B is an exemplary illustration of the dental mirror of FIG. 1A with a mirror head thereof being rotated, according to an embodiment of the present disclosure;

FIG. 2A is an exemplary illustration of a first side of a double-sided mirror of the dental mirror, according to an embodiment of the present disclosure;

FIG. 2B is an exemplary illustration of a second side of the double-sided mirror of the dental mirror, according to an embodiment of the present disclosure;

FIG. 3 is a side view of the dental mirror illustration components of a base according to an embodiment of the present disclosure;

FIG. 4 is an exemplary illustration of the dental mirror with a lighting element being turned on, according to an embodiment of the present disclosure;

FIG. 5A is a perspective view of the dental mirror according to another embodiment of the present disclosure; and

FIG. 5B is an exemplary illustration of the dental mirror of FIG. 5A with the mirror head thereof being rotated, according to an embodiment of the present disclosure.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of the present disclosure are directed to a dental mirror. The dental mirror is used by dentists or other dental professionals in performing typical dental treatment procedures within a patient's mouth (oral cavity). The dental mirror of the present disclosure eases the process of working within the oral cavity and enhances the productivity of the dentists or other dental professionals. The dental mirror includes a nano-ceramic coating that exhibits self-cleaning properties, a double-sided mirror equipped with a self-rotating feature to allow the double-sided mirror to be conveniently switched to provide a normal view or a magnified view, a rubber frame that aids comfortable retraction of the dental mirror from the oral cavity, a lighting element to emanate light within the oral cavity, and a power bank space to house a battery that renders the dental mirror portable.

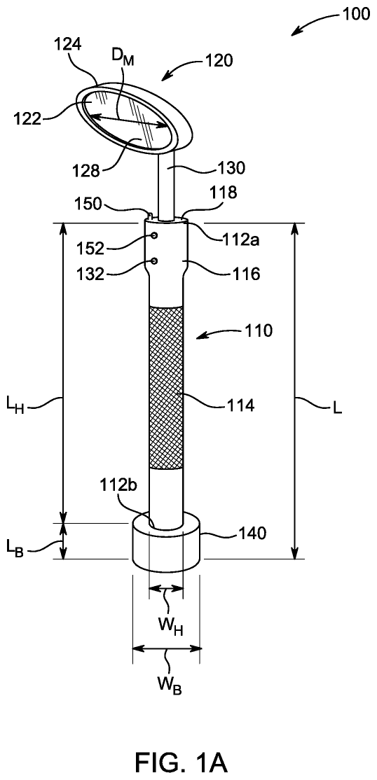

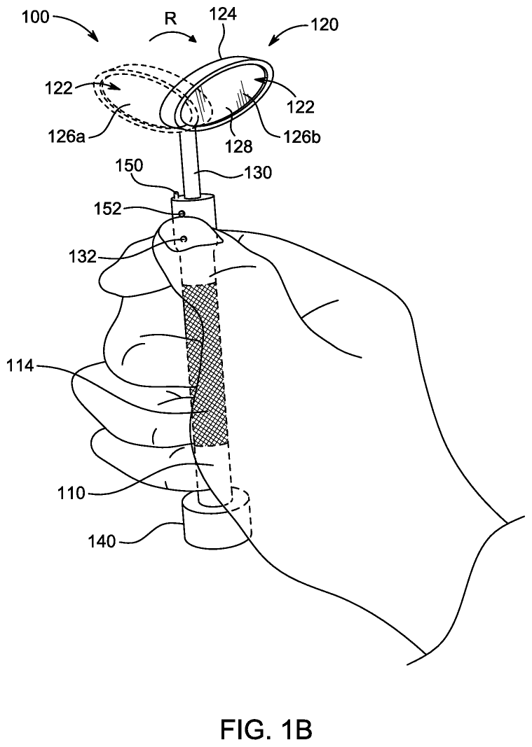

Referring to FIGS. 1A-1B, perspective views of a dental mirror 100 are illustrated. The dental mirror 100 includes a handle 110. As shown, the handle 110 is embodied as an elongated cylindrical member and defines a first end 112a and a second end 112b. In other embodiments, the handle 110 may be embodied to include a triangular, rectangular, or other suitable cross-sectional shape without any limitations.

In an embodiment, the handle 110 may include a grip region 114 extending along a portion of a length thereof. In one or more examples, the grip region 114 may be defined on a surface of the handle 110 by forming a texture pattern or a protrusion pattern, or the like. Further, in an example, the handle 110 may include an enlarged section 116, at the first end 112a thereof, having a diameter greater than a remaining portion of the handle 110. The enlarged section 116 may be configured to house one or more components of the dental mirror 100. Further, the enlarged section 116 defines a top surface 118 configured to support or allow mounting of other components of the dental mirror 100.

The dental mirror 100 also includes a mirror head 120 coupled to the first end 112a of the handle 110, where manipulation of certain elements in the handle 110 allows maneuvering of the mirror head 120 with respect to a longitudinal axis of the handle 110. As shown, the mirror head 120 is generally flat. In other embodiments, one or two sides of the mirror head 120 can be curved, for example being concave or bent inward to provide a magnified view, which will be explained in detail later. In some examples, the mirror head 120 may be circular or elliptical in shape. In an aspect of the present disclosure, the mirror head 120 is removable. For this purpose, the mirror head 120 may be attached to the handle 110 by a detachable joint (not shown), to allow removal and replacement of the mirror head 120 from the handle 110 when required. In an example, the detachable joint may be one of a form-closed joint, a screw-type joint, a spring-type joint, a pin-type joint, a plug-type joint, a keyed joint, a clamped joint or a combination thereof, as known in the art.

The mirror head 120 includes a mirror frame 124 and a double-sided mirror 122 snugly fitted to the mirror frame 124. Preferably, the double-sided mirror 122 conforms to the shape of the mirror frame 124. The double-sided mirror 122 has a diameter ‘D_{M}’ (as represented in FIG. 1A) in a range of about 20 mm to about 24 mm. In some embodiments, the diameter ‘D_{M}’ of the double-sided mirror 122 may vary between 15 mm to 30 mm. Preferably, the double-sided mirror 122 has a diameter ‘D_{M}’ of about 22 mm.

The dental mirror 100 further includes a rubber frame along a circumference of the double-sided mirror 122. In one embodiment, the mirror frame 124 may be made of rubber material and hence may be constitute the rubber frame. Hereinafter, the “mirror frame 124” has been interchangeably referred to as “rubber frame 124”. In another embodiment, the rubber frame may be a separate component configured to extend circumferentially on the double-sided mirror 122. In such an arrangement, the rubber frame may lie sandwiched between the mirror frame 124 and the double-sided mirror 122 in an assembled condition of the mirror head 120. Such use of soft and elastic material for the rubber frame 124 may help with easy insertion and retraction of the dental mirror 100 from the oral cavity of the patient, without causing injury to the teeth or other regions of the oral cavity when the dental mirror 100 accidently contacts such regions during insertion or retraction of the dental mirror 100.

Referring to FIG. 1B, the double-sided mirror 122 has a first side 126a and a second side 126b. Each of the first side 126a and the second side 126b provides a reflective surface. In an example, the reflective surfaces for the first side 126a and the second side 126b may be formed by a layer of metal(s) like silver, tin, nickel, or chromium, deposited by a wet process; or aluminum, deposited by sputtering or evaporation in vacuum. In another example, the reflective surfaces for the first side 126a and the second side 126b may be formed by one or more layers of transparent materials with suitable indices of refraction. Such techniques for forming the reflective surfaces for mirrors or the like are well known in the art and thus are not described herein for the brevity of the present disclosure.

According to aspects of the present disclosure, the mirror head 120 includes a nano-ceramic coating 128 that is hydrophobic and transparent. Specifically, the nano-ceramic coating 128 may be applied to both the first side 126a and the second side 126b of the double-sided mirror 122. The hydrophobic characteristic of the nano-ceramic coating 128 provides an effective composition for repelling spray, such as water from the surfaces of the first side 126a and the second side 126b of the double-sided mirror 122. Further, transparent characteristic of the nano-ceramic coating 128 ensures proper visibility of regions of interest on the reflective surfaces of the first side 126a and the second side 126b of the double-sided mirror 122.

In an embodiment, the nano-ceramic coating 128 includes more than 80 wt % (percentage by weight) of silicon dioxide nanoparticles having an average diameter of less than 100 nm. That is, the major constituent of the nano-ceramic coating 128 is the silicon dioxide (SiO₂) which is an inorganic oxide. In some embodiments, the percentage of silicon dioxide nanoparticles in the nano-ceramic coating 128 may be as high as 95 wt %. In particular, the percentage of silicon dioxide nanoparticles in the nano-ceramic coating 128 may vary between 80 wt % to 90 wt %, preferably between 83 wt % to 87 wt %. Further, in the present examples, the average diameter of the silicon dioxide nanoparticles may vary between 60 nm to 100 nm, preferably between 70 nm to 90 nm, preferably between 75 nm to 85 nm. In particular, the average diameter of the silicon dioxide nanoparticles may be about 80 nm.

High thermal resistance and strength of silicon dioxide in the nano-ceramic coating 128 can provide protection against environmental wear and tear on the double-sided mirror 122. For example, the nano-ceramic coating 128 may have a high melting point of about 2,900° F. (or about 1,600° C.). Additionally, the nano-ceramic coating 128 may have a molecule hardness of about 7 units and can reach up to 9 units on the Mohs scale. In an example, the nano-ceramic coating 128 may be formed by suspending silicon dioxide in the form of nanoparticles in a resin, that may be applied as paint. Preferably, the nano-ceramic coating 128 is formed by treating the double-sided mirror 122 with KubeBond GlassShield. The KubeBond GlassShield is a product known in the art to have the composition as described in the preceding paragraph. The KubeBond GlassShield is in the form of “liquid ceramic” which may transform from a transparent liquid to a transparent ceramic film when applied on surfaces, such as the reflective surfaces of the first side 126a and the second side 126b of the double-sided mirror 122, making it easier to be implemented for the dental mirror 100.

According to some aspects of the disclosure, in the nano-ceramic coating 128, a ceramic barrier seals an underlying surface against contact with the environment and provides hydrophobic and oleophobic properties, scratch resistance, anti-stain functionality and acid/alkaline resistance. The ceramic barrier enables strong protection and creates a surface that stays cleaner for longer, without any visible finishing after application. The described composition for the nano-ceramic coating 128 provides high hydrophobicity and prevents adhesion of liquids, while enhancing the shine and color of the reflective surfaces of the first side 126a and the second side 126b of the double-sided mirror 122. With the super hydrophobic effect, the nano-ceramic coating 128 reduces the surface energy of the aforementioned reflective surfaces with a water contact angle of 110-120 degrees, allowing water to bead together and easily roll-off therefrom. Further, such composition of the nano-ceramic coating 128 exhibits excellent bonding strength with the coated surface and forms a hard-cubic ceramic layer, which protects the coated surfaces from contaminants and harsh chemicals, without affecting the look or feel of the reflective surfaces, thus making the double-sided mirror 122 strong, shiny, and sparkling, and easy to maintain. Further, such composition can withstand high pressure (for example, up to 30 PSI), high temperature sterilization (for example, up to 274° F.), and prevents scratching or chipping of the reflective surfaces that may otherwise occur during autoclaving (as required for surgical instruments) without warping or melting, thus proving no harmful health-related side effects, and making it ideal for the dental mirror 100.

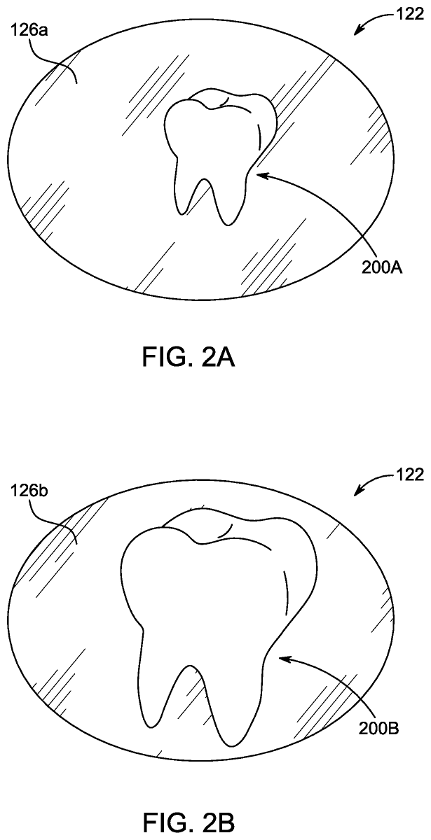

Further, in an aspect of the present disclosure, as illustrated in FIGS. 2A and 2B, the first side 126a of the double-sided mirror 122 provides a normal view (represented by the reference numeral 200A) and the second side 126b of the double-sided mirror 122 provides a magnified view (represented by the reference numeral 200B). For this purpose, the first side 126a of the double-sided mirror 122 may be embodied as a plane mirror (as known in the art) which forms reflected images in their normal proportions, and the second side 126b of the double-sided mirror 122 may be embodied as a concave mirror (as known in the art) which forms reflected images in their magnified proportions, such as the magnified view 200B. Further, with the concave mirror, the second side 126b may refract some light thereby rendering the magnified view 200B of the oral cavity larger, brighter, and not inverted, and thus improving visibility for the user of the dental mirror 100. In an example, the double-sided mirror 122 may be configured to provide between 2 times (2×) magnification to 6 times (6×) magnification when switching from the normal view 200A to the magnified view 200B. In particular, the second side 126b can provide four times (4×) magnification.

Typically, size of tooth surface and indications of early signs of harm to a tooth are difficult to detect and view in detail with only the normal view 200A as reflected by the first side 126a of the double-sided mirror 122. In order to clearly view an area suspected of damage in the oral cavity and to accurately diagnose issues caused to the teeth, the user of the dental mirror 100 may need magnification of the suspected area. To that end, the magnified view 200B as reflected by the second side 126b of the double-sided mirror 122 assists the user to effectively view the suspected area and perform the dental treatment procedure. Further, as may be appreciated, the user may need to switch between the normal view 200A and the magnified view 200B to properly assess condition of the oral cavity while performing the dental treatment procedure.

The dental mirror 100 also includes an electrical or mechanical component for rotating the mirror head 120. As illustrated in FIG. 1B, such structure for rotating the mirror head 120 (hereinafter referred to as “rotation source”) may function to dispose the double-sided mirror 122 between a first position (as depicted in FIG. 1A and represented by broken lines in FIG. 1B) and a second position (as represented by solid lines in FIG. 1B). The term “first position” refers to a position of the first side 126a of the double-sided mirror 122 facing the suspected area in the oral cavity to provide the normal view 200A thereof and the term “second position” refers to a position of the second side 126b of the double-sided mirror 122 facing the suspected area in the oral cavity to provide the magnified view 200B thereof. As such, the user may conveniently switch between the first side 126a and the second side 126b while performing the dental treatment procedure.

In an embodiment, the rotation source may be located in the handle 110. Specifically, the rotation source may be accommodated in the enlarged section 116 of the handle 110. In particular, as shown in FIGS. 1A and 1B, the mirror head 120 includes a shaft 130 arranged between the double-sided mirror 122 and the handle 110. As used herein, the term “arranged between” may be alternatively understood as “extending between” or “coupled between”. For example, the shaft 130 extends between the double-sided mirror 122 and the handle 110. Alternatively, the ends of the shaft 130 may be operably coupled to the double-sided mirror 122 and the handle 110, respectively. As used herein, the term “operably coupled” may include the means for rotating the mirror head 120. The shaft 130 may function as an intermediate member to attach the mirror head 120 to the handle 110. In the illustrated embodiment, the shaft 130 is coupled to the first end 112a of the handle 110 such that the shaft 130 is rotatable and removable. In an example, the shaft 130 may be coupled to the handle 110 by a quick disconnect ball joint as known in the art, to allow for quick disconnect (removing) of the shaft 130 from the handle 110. Further, the shaft 130 may be rotatably connected to the rotation source disposed in the handle 110, such that the shaft 130, and thereby the mirror head 120, may rotate along a rotational direction “R” (as shown in FIG. 1B) with respect to the handle 110.

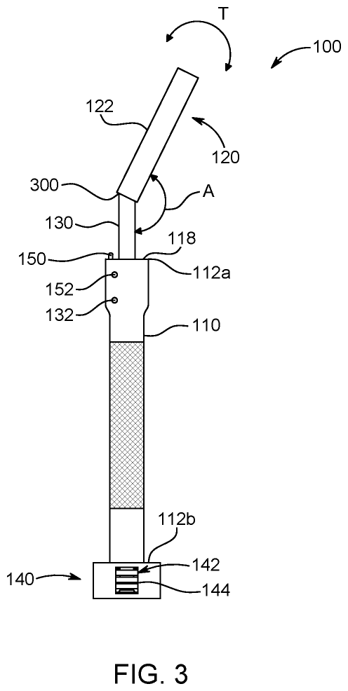

Referring to FIG. 3, the shaft 130 and the double-sided mirror 122 are placed at an angle ‘A’ such that the shaft 130 and the double-sided mirror 122 are non-coplanar. In an example, the shaft 130 may be in the form of a gooseneck or “C-shape” so that the double-sided mirror 122 is disposed at the angle ‘A’ with respect to the handle 110. In another example, the double-sided mirror 122 may be manually rotatable on the shaft 130 for adjusting the angle ‘A’. For this purpose, the double-sided mirror 122 may be connected to the shaft 130 in the mirror head 120 using a rotational joint 300 to provide a rotational movement of the double-sided mirror 122 with respect to the shaft 130 along a tilting direction “T” (as shown). The rotational joint 300 may be any one of a pivot joint, a hinge joint, a ball and socket joint as known in the art for providing uniaxial, biaxial, or multiaxial rotational movement. Such an arrangement allows the user to manually adjust the angle ‘A’ of the double-sided mirror 122 with respect to the shaft 130 in the dental mirror 100, as may be required for performing the dental treatment procedure. It will be understood that the rotational joint 300 will be located internally in the components and hence, for the brevity in figures, the connection portion between the shaft 130 and the mirror head 120 is referenced as the rotational joint 300 in FIG. 3.

The shaft 130 may be rotatably coupled to the rotation source. In an example, the rotation source may include a stepper motor operably connected to the shaft 130 at the first end 112a of the handle 110. The stepper motor may be connected to the shaft 130 by a worm drive to provide discrete rotational movement to the shaft 130, thus allowing adjustment of orientation of the mirror head 120 with respect to the handle 110. In some examples, the dental mirror 100 may include roller bearings (not shown) provided at an interface of the stepper motor and the shaft 130 to provide smooth rotational movement thereof. In some embodiments, the dental mirror 100 may be configured to allow extension of length of the shaft 130 with respect to the handle 110.

Further, the dental mirror 100 includes a first button 132. As better shown in FIG. 1B, the first button 132 may be located on the handle 110 proximal to the first end 112a thereof, such that a thumb of the user may rest in vicinity of the first button 132 for easy reach. The first button 132 is configured to control rotation of the mirror head 120. For example, “rotation” can include moving or pressing the first button 132 on the handle 110 so that the mirror head 120 switches from the first side 126a (e.g., low power magnification) to the second side 126b (e.g., high power magnification), or vice versa. Specifically, the first button 132 is configured to control rotation of the shaft 130. In particular, the first button 132 may generate a control signal for controlling the rotational movement provided by the stepper motor. In an embodiment, a single press of the first button 132 may generate the control signal to cause the stepper motor to provide a discrete rotational movement output, and therefore result in a discrete rotation of the shaft 130 and thus the mirror head 120. In an embodiment, the shaft 130 rotates 180 degrees per rotation to switch sides (such as the first side 126a and the second side 126b) for the double-sided mirror 122. In other words, for each single press of the first button 132, the stepper motor may be configured to provide a discrete 180 degrees rotational movement output, which results in 180 degrees rotation of the mirror head 120, and thus switching from the first side 126a to the second side 126b, thereby changing the view of the suspected area from the normal view 200A to the magnified view 200B, or vice versa. In some examples, the rotation source may alternatively be implemented by a regular motor with limit switches to achieve the same functionality. In other examples, the discrete rotational movement output provided by the rotation source may not be 180 degrees but may be converted to 180 degrees rotation of the shaft 130 by employing one or more gears or the like. In some embodiments, each press of the first button 132 may cause rotation of the mirror head 120 by an angle corresponding to a duration of the press. As such, the first button 132 may be pressed multiple times to change the sides of the double-sided mirror 122. As such, the user may orient the double-sided mirror 122 at a desired angle to clearly view the suspected area within the oral cavity. Such configurations and other alternatives may be contemplated by a person skilled in the art and thus have not been explained in detail herein for the brevity of the present disclosure. In some embodiments, accessing the first button 132 may also allow extension of length of the shaft 130 as described earlier.

Referring back to FIGS. 1A-1B, in combination with FIG. 3, as illustrated, the dental mirror 100 further includes a base 140 affixed to the second end 112b of the handle 110 and wider than the handle 110. In an embodiment, the base 140 is 1.5 to 2.3 times as wide as the handle 110. In another embodiment, the base 140 may be 1.8 to 2 times as wide as the handle 110. In some embodiments, the base 140 may have a cross-section similar to that of the handle 110. In the illustrated embodiment, the handle 110 and the base 140 each include a circular cross-section. In some embodiments, the base 140 has a diameter ‘W_{B}’ (see FIG. 1A) in a range of about 9 mm to about 14 mm and the handle 110 has a diameter ‘W_{H}’ in a range of about 4 mm to about 8 mm. In another embodiment, the base 140 may have the diameter ‘W_{B}’ in a range of about 10 mm to about 12 mm, preferably about 11 mm, and the diameter ‘W_{H}’ of the handle 110 may be in a range of about 5 mm to about 7 mm, preferably about 6 mm.

Further, the base 140 may be shorter than the handle 110. That is, a length ‘L_{B}’ the base 140 is smaller than a length ‘L_{H}’ of the handle 110. In an embodiment, an overall length ‘L’ of the base 140 and the handle 110 is in a range of about 130 mm to about 150 mm. The overall length ‘L’ represents a sum of the length ‘W_{H}’ of the handle 110 and the length ‘L_{B}’ the base 140. Generally, the overall length ‘L’ of the base 140 and the handle 110 may vary from about 100 mm to about 200 mm. In some embodiments, the overall length ‘L’ of the base 140 and the handle 110 is about 140 mm.

In an embodiment, the dental mirror 100 further includes a battery bank space 142 (as shown in FIG. 3) defined in the base 140. In general, the base 140 may be a hollow member configured to define the battery bank space 142 adapted to accommodate a battery 144 therein, which may power electrical components of the dental mirror 100. For instance, the battery 144 may be electrically connected to the rotation source (such as the stepper motor) in the enlarged section 116 of the handle 110 using electrical wires routed through the length ‘L_{H}’ of the handle 110. Provision of the battery 144 renders the dental mirror 100 portable and eliminates dependency on an electrical socket.

In some examples, the battery 144 may be in the form of a button cell, also referred to as a button battery or a coin battery. The base 140 may include a charging interface (not shown) to allow for recharging the battery 144. The battery bank space 142 may define a volume to accommodate a sufficient number of batteries to power the dental mirror 100 for a long duration.

Further, it may be appreciated that the inclusion of the double-sided mirror 122 in the mirror head 120 and the battery 144 in the battery bank space 142 in the base 140 adds weight at different sections in the dental mirror 100. In the dental mirror 100 of the present disclosure, masses of the mirror head 120 and the base 140 are balanced so that the dental mirror 100 has a center of gravity which is closer to the base 140 than to the mirror head 120. Since the dental mirror 100 is held at the handle 110, the center of gravity of the dental mirror 100 may preferably be located in the handle 110 (thus closer to the base 140 than to the mirror head 120) to allow for convenient handling of the dental mirror 100 by the user. Typically, an overall mass of the base 140 and the handle 110 may be greater than a mass of the double-sided mirror 122, thus the dental mirror 100 may be easily handled by the user. With such configuration, the dental mirror 100 may be able to stand upright when the base 140 is supported on a planar surface, as may be desired for storage or display purposes of the dental mirror 100.

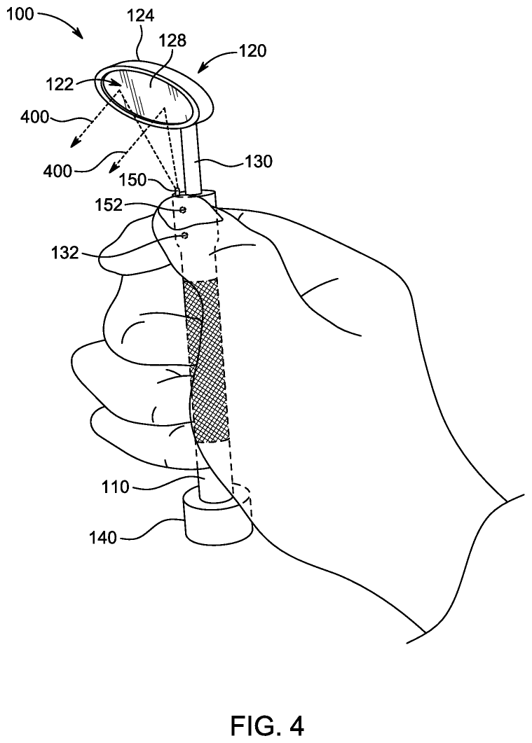

In an embodiment, the dental mirror 100 further includes a lighting element 150 for producing light. In a preferred embodiment, the lighting element 150 is positioned on the handle 110 and faces the mirror head 120. In particular, the lighting element 150 may be positioned on the top surface 118 of the handle 110. In the illustrated examples, the lighting element 150 is shown as a point light source, such as a single LED. As better shown in FIG. 4, the lighting element 150 may be oriented such that light rays 400 generated by the lighting element 150 may be reflected by the double-sided mirror 122 of the mirror head 120, to illuminate a desired area in the oral cavity during the dental treatment procedure. In other examples, the lighting element 150 may be in the form of a light strip (such as an LED strip) which may surround a circumference of the double-sided mirror 122. As shown, the mirror frame 124 provides flat surface around the circumference of the double-sided mirror 122. The lighting element 150 may be arranged on the flat surface of the mirror frame 124, to be disposed on the circumference of the double-sided mirror 122. Further, as illustrated, the dental mirror 100 may also include a second button 152 configured to turn on and off the lighting element 150. As better shown in FIG. 4, the second button 152 may be located on the handle 110 proximal to the first end 112a thereof, such that a thumb of the user can easily reach the second button 152.

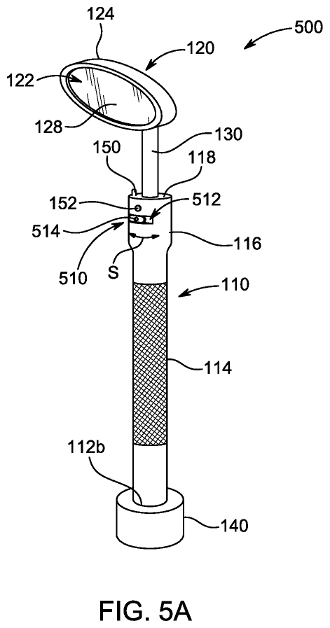

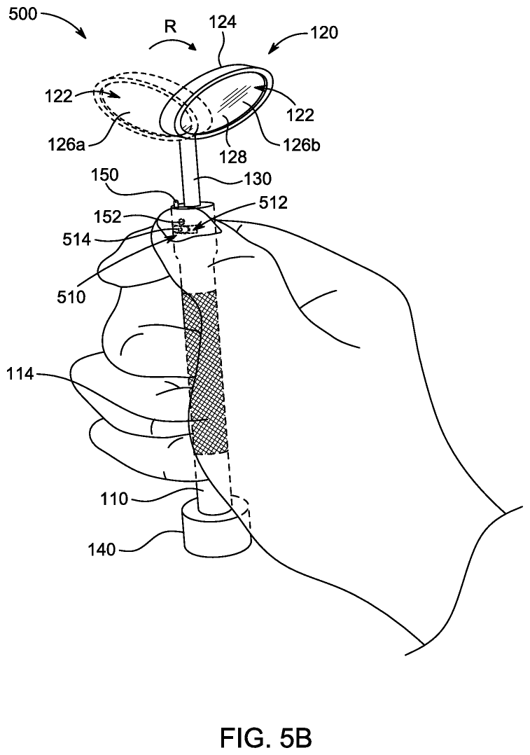

FIGS. 5A and 5B illustrates a dental mirror 500 according to another embodiment of the present disclosure. The dental mirror 500 may generally be similar in construction to the dental mirror 100 described above. The dental mirror 500 includes a slot 512 with a slider 514 disposed therein, which together constitutes a first button 510. As shown, the slot 512 may be defined to extend along a portion of a circumference of the handle 110, proximal to the first end 112a thereof. Further, the slider 514 may be adapted to move along a sliding direction “S” (as shown in FIG. 5A). The slider 514 may be mechanically connected to the double-sided mirror 122 via the shaft 130 to cause rotation of the double-sided mirror 122 by thumb action (as depicted in FIG. 5B). Connection between the double-sided mirror 122 and the slider 514 may be configured such that the movement of the slider 514 along the entire length of the slot 512 may cause 180 degrees rotation of the double-sided mirror 122, and thus switching from the first side 126a providing the normal view 200A to the second side 126b providing the magnified view 200B, or vice-versa. In some embodiments, movement of the slider 514 to a halfway along the length of the slot 512 may rotate the double-sided mirror 122 by 180 degrees, thereby switching the sides of the double-sided mirror 122. Further movement of the slider 514 to cover remaining half of length of the slot 512 may rotate the double-sided mirror 122 again by 180 degrees, thereby bringing the double-sided mirror 122 to initial position. In these embodiments, the slider 514 may be located at any desired position along the length of the slot 512, to achieve a desired rotation angle of the double-sided mirror 122.

To this end, embodiments of the present disclosure provide the dental mirrors 100 and 500 that include features of self-cleaning, lighting the oral cavity, rotating the sides of the double-sided mirror 122, and magnifying the suspected area. Additionally, the dental mirrors 100 and 500 provide a continuous sight of the suspected area by reflecting clear images on the sides of the double-sided mirror 122. The dental mirror 100, 500 can be manufactured easily in a cost-efficient manner.

To conform to surgical requirements, the dental mirrors 100 and 500 can be constructed from non-corroding, non-pressure sensitive, and non-heat sensitive materials. In one or more examples, the components of the dental mirrors 100 and 500, including the handle 110, the mirror head 120 (specifically the mirror frame 124 therein) and the base 140 may be made of metallic material, such as titanium alloy. Use of such material allows the dental mirrors 100 and 500 to be autoclaved, as required with the surgical instruments, without adverse effects.

The dental mirrors 100 and 500 of the present disclosure provide a self-cleaning feature with the use of the nano-ceramic coating 128 which has hydrophobic properties and thus eliminates settlement of water particles or debris on the reflective surfaces of the dental mirrors 100 and 500. This allows cleaning the double-sided mirror 122 while still in use, thereby eliminating the need to pause the dental treatment procedure repeatedly for manual cleaning. As such, the dental mirrors 100 and 500 achieve significant time saving for dental professionals in completing the dental treatment procedures. Further, the dental mirrors 100 and 500 do not require a complete redesign unlike traditional dental mirrors which requires widening of handle to allow water and air channels to be routed therethrough to achieve the self-cleaning feature.

The dental mirrors 100 and 500 reduce the need of using multiple tools while working, by providing multiple aids therein. In addition to the self-cleaning feature, the dental mirrors 100 and 500 provide two reflective surfaces (providing the normal view 200A and the magnified view 200B, as desired) as part of a removable and replaceable mirror head 120. The dental mirrors 100 and 500 are also provided with buttons 132, 152 for rotating the mirror head 120 and turning on and off the lighting element 150 for convenience of the user. The reflective surface of the mirror head 120 is aligned with the buttons 132, 152 such that the reflective surface is oriented properly to allow the user holding the handle 110 to have a direct view of the reflective surface.

Embodiments of the present disclosure are illustrated with respect to FIG. 1A through FIG. 5B. The embodiments describe the dental mirrors 100 and 500, including the handle 110; the mirror head 120 affixed to the first end 112a of the handle 110, where the mirror head 120 includes the double-sided mirror 122 having the nano-ceramic coating 128 that is hydrophobic and transparent. The nano-ceramic coating 128 includes more than 80 wt % of silicon dioxide nanoparticles having the average diameter of less than 100 nm. The dental mirrors 100 and 500 also include means for rotating the mirror head 120; and the first button 132, 510 configured to control rotation of the mirror head 120.

Obviously, numerous modifications and variations of the present disclosure are possible in light of the above teachings. It is therefore to be understood that within the scope of the appended claims, the invention may be practiced otherwise than as specifically described herein.

1. A dental mirror, comprising:
a handle;
a mirror head coupled to a first end of the handle, the mirror head including a double-sided mirror having a nano-ceramic coating that is hydrophobic and transparent, wherein the nano-ceramic coating comprises more than 80 wt % of silicon dioxide nanoparticles having an average diameter of less than 100 nm;
means for rotating the mirror head; and
a first button configured to control rotation of the mirror head.

2. The dental mirror of claim 1, wherein the nano-ceramic coating is formed by treating the double-sided mirror with KubeBond GlassShield.

3. The dental mirror of claim 1, wherein the double-sided mirror has a first side providing a normal view and a second side providing a magnified view.

4. The dental mirror of claim 3, wherein the second side provides four times magnification.

5. The dental mirror of claim 1, further comprising:
a lighting element for producing light; and
a second button configured to turn on and off the lighting element.

6. The dental mirror of claim 5, wherein the lighting element is positioned on the handle and faces the mirror head.

7. The dental mirror of claim 1, wherein the mirror head is removable.

8. The dental mirror of claim 1, further comprising a base affixed to a second end of the handle, the base being wider than the handle.

9. The dental mirror of claim 8, wherein the base is 1.5 to 2.3 times as wide as the handle.

10. The dental mirror of claim 9, wherein:
the base has a diameter of about 9 mm to about 14 mm, and
the handle has a diameter of about 5 mm to about 7 mm.

11. The dental mirror of claim 8, wherein an overall length of the base and the handle is in a range of about 130 mm to about 150 mm.

12. The dental mirror of claim 8, wherein masses of the mirror head and the base are balanced so that the dental mirror has a center of gravity which is closer to the base than to the mirror head.

13. The dental mirror of claim 8, further comprising:
a battery bank space defined in the base.

14. The dental mirror of claim 1, wherein the double-sided mirror has a diameter in a range of about 20 mm to about 24 mm.

15. The dental mirror of claim 1, wherein the mirror head includes a shaft arranged between the double-sided mirror and the handle.

16. The dental mirror of claim 15, wherein:
the shaft is coupled to the first end of the handle such that the shaft is rotatable and removable, and
the first button is configured to control rotation of the shaft.

17. The dental mirror of claim 16, wherein:
the shaft rotates 180 degrees per rotation to switch sides for the double-sided mirror.

18. The dental mirror of claim 15, wherein the shaft and the double-sided mirror are placed at an angle such that the shaft and the double-sided mirror are non-coplanar.

19. The dental mirror of claim 18, wherein the double-sided mirror is manually rotatable on the shaft for adjusting the angle.

20. The dental mirror of claim 1, further comprising:
a rubber frame along a circumference of the double-sided mirror.
